(19) United States
(12) Patent Application Publication
(10) Pub. No.: US 2019/0231214 A1
(43) Pub. Date: Aug. 1, 2019

(54) ELECTROCARDIOGRAPH AND ELECTROCARDIOGRAPH SYSTEM

(71) Applicant: SOCIONEXT INC., Yokohama-shi (JP)

(72) Inventor: Ryusuke KURACHI, Yokohama (JP)

(21) Appl. No.: 16/195,217

(22) Filed: Nov. 19, 2018

(30) Foreign Application Priority Data
Jan. 31, 2018 (JP) 2018-014423

(51) Int. Cl. A61B 5/0428; A61B 5/044; A61B 5/0432

(52) U.S. Cl. CPC A61B 5/04288; A61B 5/0432; A61B 5/044

(57) ABSTRACT

An electrocardiograph is disclosed. A first electrocardiograph measures a first electrocardiogram waveform with a potential of a first electrode being as a reference potential among a plurality of electrodes. A second electrocardiograph measures a second electrocardiogram waveform with a potential of a second electrode being as the reference potential among the plurality of electrodes. The first electrocardiograph and the second electrocardiograph are switched in a time sharing manner, and the first electrocardiogram waveform and the second electrocardiogram waveform are measured.

### CROSS-REFERENCE TO RELATED APPLICATIONS

This application is based on and claims the benefit of priority of the prior Japanese Patent Application No. 2018-014423, filed on Jan. 31, 2018, the entire contents of which are incorporated herein by reference.

### BACKGROUND OF THE INVENTION

### 1. Field of the Invention

The disclosures herein relate to an electrocardiograph and an electrocardiograph system.

### 2. Description of the Related Art

Conventionally, an electrocardiograph is known to acquire electrocardiogram waveforms by limb leads performed by attaching four electrodes to respective limbs. In the case of acquiring the electrocardiogram waveforms of a person with this electrocardiograph, measurement is usually performed by pinching each of limbs (wrists and ankles) with a clip type electrode or the like.

Also, in a case of measuring the electrocardiogram waveforms of an animal by the limb lead, in order to perform measurement simply without restraining the animal, a method is known of measuring the electrocardiogram waveforms in a state in which the limbs are placed respectively on four electrode plates.

### RELATED-ART DOCUMENTS

### Patent Document

### [Patent Document 1] Japanese Laid-open Patent Publication No. 2008-237379

### [Patent Document 2] Japanese Laid-open Patent Publication No. 2016-174839

[Patent Document 3] International Publication Pamphlet No. WO2011/018855
[Patent Document 4] U.S. Pat. No. 6,445,941

### SUMMARY OF THE INVENTION

According to an embodiment, an electrocardiograph, including: a first electrocardiograph configured to measure a first electrocardiogram waveform with a potential of a first electrode being set as a reference potential among a plurality of electrodes; and a second electrocardiograph configured to measure a second electrocardiogram waveform with a potential of a second electrode being set as the reference potential among the plurality of electrodes, wherein the first electrocardiograph and the second electrocardiograph are switched in a time sharing manner, and the first electrocardiogram waveform and the second electrocardiogram waveform are measured.

### BRIEF DESCRIPTION OF THE DRAWINGS

Other objects and further features of the present invention will be apparent from the following detailed description when read in conjunction with the accompanying drawings, in which:

FIG. 1 is a diagram for explaining an electrocardiograph (ECG) system in a first embodiment;

FIG. 2 is a flowchart for explaining an operation of the electrocardiograph system of the first embodiment;

FIG. 3A through FIG. 3D are first diagrams for explaining a measurement of electrocardiogram waveforms by the electrocardiograph system of the first embodiment;

FIG. 4A through FIG. 4D are second diagrams for explaining the measurement of the electrocardiogram waveforms by the electrocardiograph system of the first embodiment;

FIG. 5 is a diagram illustrating a display example of the electrocardiograph system of the first embodiment;

FIG. 6 is a diagram for explaining an electrocardiograph system of a second embodiment;

FIG. 7 is a diagram illustrating an example of a normal electrocardiogram waveform;

FIG. 8 is a diagram illustrating a display example of the electrocardiograph system of the second embodiment; and

FIG. 9 is a diagram for explaining an electrocardiograph system of a third embodiment.

### DESCRIPTION OF THE PREFERRED EMBODIMENTS

In a case of measuring electrocardiogram waveforms of an animal by using electrode plates, all limbs of the animal may unstably contact the electrode plates. For instance, among four electrode plates, three electrode plates alone may contact three of the limbs. In addition, limbs contacting the electrode plates change depending on a movement of the animal (a living body).

Also, when a limb does not contact a corresponding electrode, that is to be a reference, among the four electrode plates, all lead data acquired by the limb leads may be effected.

In the following, embodiments of the invention will be described with reference to the accompanying drawings. The embodiments described below realize to reduce an effect to the electrocardiogram waveforms due to an unstable contact between the electrode plates and the living body.

### First Embodiment

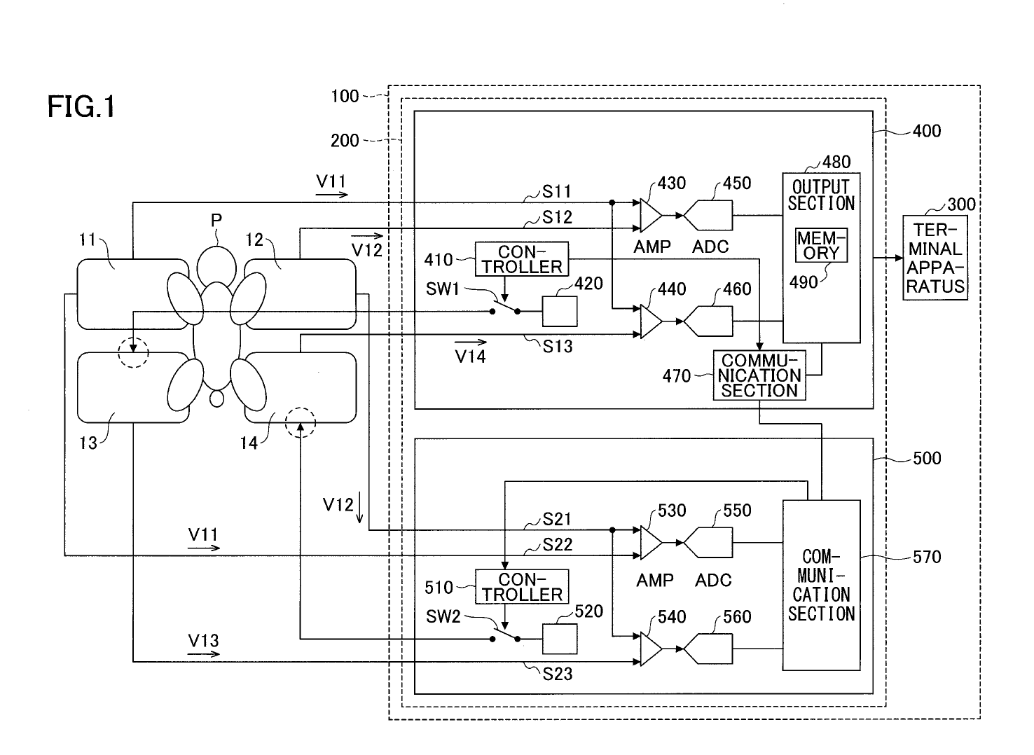

A first embodiment will be described with reference to drawings. FIG. 1 is a diagram for explaining an electrocardiograph (ECG) system in the first embodiment.

An electrocardiograph system 100 in the first embodiment includes an electrocardiograph 200 and a terminal apparatus 300. The electrocardiograph 200 is connected to electrode plates 11, 12, 13, and 14, measures the electrocardiogram waveforms of a living body P with four limbs placed respectively on the electrode plates 11 to 14, and outputs the electrocardiogram waveforms to the terminal apparatus 300. The four limbs are regarded as two pairs of legs of a vertebrate; that is, front legs and hind legs in a case of an animal, and both hands and both feet in a case of a human being. Also, for instance, the electrode plates 11 to 14 in the first embodiment may be metal plates or the like.

For instance, the terminal apparatus 300 may be a tablet type terminal apparatus or a general computer, and displays the electrocardiogram waveforms output from the electrocardiograph 200.

The electrocardiograph 200 of the first embodiment includes an electrocardiograph 400 and an electrocardiograph 500. In other words, the electrocardiograph 200 is regarded as one substrate on which the electrocardiograph 400 and the electrocardiograph 500 are mounted. The electrocardiograph 400 operates as a master of the electrocardiograph 500, and the electrocardiograph 500 operates as a slave of the electrocardiograph 400.

The electrocardiograph 400 and the electrocardiograph 500 alternately applies a reference potential to a different electrode plate among the electrode plates 11 to 14, and acquires the electrocardiogram waveforms of the living body P by the limb lead. The limb lead corresponds to a method for acquiring the electrocardiogram waveforms (bioelectrical signals) emanating from a heart of the living body P, in which four electrodes are attached to the four limbs of the living body P.

More specifically, in the electrocardiograph system 100, first limb 6-lead waveforms for the four limbs are acquired by the electrocardiograph 400 where a potential of the electrode plate 13 is set as the reference potential, and second limb 6-lead waveforms for the four limbs are acquired by the electrocardiograph 500 where a potential of the electrode plate 14 is set as the reference potential.

The first limb 6-lead waveforms, in which the potential of the electrode plate 13 is set as the reference potential, are depicted as a first lead waveform I, a second lead waveform II, a third lead waveform III, a fourth lead waveform aVR, a fifth lead waveform aVL, and a sixth lead waveform aVF. Each of the first through sixth lead waveforms is obtained by the following expressions, where the potential of the electrode plate 11 is denoted by a potential V11, the potential of the electrode plate 12 is denoted by a potential V12, and a potential of the electrode plate 14 is denoted by a potential V14:

the first lead waveform I=V12−V11,

the second lead waveform II=V14−V11,

the third lead waveform III=II−I,

the fourth lead waveform aVR=−(II+I)/2,

the fifth lead waveform aVL=I−(II/2), and

the sixth lead waveform aVF=II−(I/2).

In addition, the second limb 6-lead waveforms, in which the potential of the electrode plate 14 is set as the reference potential, are depicted as a first lead waveform I′, a second lead waveform II′, a third lead waveform III′, a fourth lead waveform aVR′, a fifth lead waveform aVL′, and a sixth lead waveform aVF′. Each of the first through sixth lead waveforms is acquired by the following expressions:

the first lead waveform I′=V11−V12,

the second lead waveform II′=V13−V12,

the third lead waveform III′=II′−I′,

the fourth lead waveform aVR′=−(II+I)/2,

the fifth lead waveform aVL′=I−(II/2), and

the sixth lead waveform aVF′=II−(I/2).

In the electrocardiograph system 100 of the first embodiment, the first lead waveform I and the second lead waveform II are measured by the electrocardiograph 400, and the first lead waveform I′ and the second lead waveform II′ are measured by the electrocardiograph 500, and other lead waveforms are calculated by the terminal apparatus 300.

Also, in the electrocardiograph system 100 of the first embodiment, the measurement of the electrocardiograph 400 and the measurement of the electrocardiograph 500 are conducted in a time sharing manner.

In other words, the electrocardiograph system 100 alternately applies a potential to an electrode plate to be the reference of the electrocardiograph 400 and an electrode plate to be the reference of the electrocardiograph 500, so that the electrocardiograph 400 and the electrocardiograph 500 alternately measures the electrocardiogram waveforms of the living body P. That is, the electrocardiograph 200 of the first embodiment switches between the electrocardiograph 400 and the electrocardiograph 500 in the time sharing manner, and measures the electrocardiogram waveforms of the living body P.

As described above, in the electrocardiograph system 100 of the first embodiment, by switching, in the time sharing manner, among two or more electrocardiographs, in which potentials of different electrode plates in a plurality of electrode plates are set as the reference potential, it is possible to reduce effects on electrocardiogram waveforms due to an unstable contact between the plurality of electrode plates and the living body P.

Configurations of the electrocardiograph 400 and the electrocardiograph 500 will be described below. The electrocardiograph 400 of the first embodiment includes a controller 410, a driving section 420, amplifiers 430 and 440, analog to digital converters (ADC) 450 and 460, a communication section 470, an output section 480, and a switch SW1.

The controller 410 controls ON and OFF of the switch SW1. Specifically, the controller 410 of the first embodiment outputs a control signal to the switch SW1. With respect to the control signal, a term (an ON term-period) is defined to turn on the switch SW1, and another term (an OFF term-period) is defined to turn off the switch SW1. Also, when the electrocardiograph system 100 starts to measure the electrocardiogram waveforms, the controller 410 sends a notification indicating a start of the measurement to the electrocardiograph 500 through the communication section 470.

For instance, an instruction of the start of measuring the electrocardiogram waveforms may be given to the electrocardiograph 400 by an operation made at the terminal apparatus 300, or may be given by an operation button or the like, which is provided on a housing of the electrocardiograph 200 and instructs a start or an end of measurement.

The switch SW1 connects or disconnects between the electrode plate 13 and the driving section 420. The driving section 420 applies the potential to be the reference to the electrode plate 13.

In the first embodiment, upon turning on the switch SW1, the electrode plate 13 is connected to the driving section 420, and the potential of the electrode plate 13 attains the reference potential by supplying a voltage of the electrode plate 13. Also, in the first embodiment, upon turning off the switch SW1, the electrode plate 13 is disconnected from the driving section 420, and the voltage is not supplied to the electrode plate 13. The reference potential may be determined beforehand.

The electrode plate 11 is connected to the amplifier 430 by a signal line S11, and the electrode plate 12 is connected to the amplifier 430 by a signal line S12. Moreover, the electrode plate 14 is connected to the amplifier 440 by a signal line S13.

The potential V11 of the electrode plate 11 is applied to an input terminal of the amplifier 430, the potential V12 of the electrode plate 12 is applied to another input terminal of the amplifier 430, and the amplifier 430 outputs a difference between the potential V11 of the electrode plate 11 and the potential V12 of the electrode plate 12. Accordingly, a waveform output from the amplifier 430 is the first lead waveform I. An output of the amplifier 430 is input to the ADC 450.

The ADC 450 converts a signal input from the amplifier 430 into a digital signal, and stores the digital signal in a memory 490 of the output section 480.

The potential V11 of the electrode plate 11 is applied to an input terminal of the amplifier 440, the potential V14 of the electrode plate 14 is applied to another input terminal of the amplifier 440, and the amplifier 440 outputs a difference between the potential V11 of the electrode plate 11 and the potential V14 of the electrode plate 14. Accordingly, a waveform output from the amplifier 440 is the second lead waveform II. An output of the amplifier 440 is input to the ADC 460.

The ADC 460 converts a signal input from the amplifier 440 into a digital signal, and stores the digital signal in the memory 490 of the output part 480.

Accordingly, the signal output from the ADC 450 is regarded as a signal converted from the first lead waveform I into a digital signal, and the signal output from the ADC 460 is regarded as a signal converted from the second lead waveform II into a digital signal.

The communication section 470 interfaces between the electrocardiograph 400 and the electrocardiograph 500. Specifically, the communication section 470 sends an instruction of the start of measuring the electrocardiogram waveforms and an instruction of the end of measuring the electrocardiogram waveforms, to the electrocardiograph 500. Moreover, the communication section 470 receives the digital signal output from the electrocardiograph 500 and outputs the digital signal to the output section 480.

The output section 480 includes the memory 490, stores the digital signals output from the ADC 450 and the ADC 460 and the digital signal output from the electrocardiograph 500 in the memory 490, and outputs these digital signals to the terminal apparatus 300 at a given timing.

The electrocardiograph 500 of the first embodiment includes a controller 510, a driving section 520, amplifiers 530 and 540, an ADC 550, an ADC 560, a communication section 570, and a switch SW2.

The controller 510 controls ON and OFF of the switch SW2. Specifically, the control section 510 of the first embodiment outputs a control signal to the switch SW2. With respect to the control signal, a term (an ON term-period) is defined to turn on the switch SW2, and another term (an OFF term-period) is defined to turn off the switch SW2.

Upon receiving the instruction of the start of measuring the electrocardiogram waveforms through the communication section 570, the control section 510 may control ON and OFF of the switch SW2.

The switch SW1 and the switch SW2 of the first embodiment are controlled to be ON or OFF in order for one switch to be in the ON term-period while the other switch is in the OFF term-period. The ON term-periods of the switch SW1 and the switch SW2 may be the same time length.

The switch SW2 connects or disconnects between the electrode plate 14 and the driving section 520. The driving section 520 applies the potential to be the reference to the electrode plate 14.

In the first embodiment, upon turning on the switch SW2, the electrode plate 14 is connected to the driving section 520, a voltage is supplied to the electrode plate 14, and a potential of the electrode plate 14 becomes the reference potential.

The electrode plate 12 is connected to the amplifier 530 by a signal line S21, the electrode plate 11 is connected to the amplifier 530 by a signal line S22, and the electrode plate 13 is connected to the amplifier 540 by the signal line S23.

The potential V11 of the electrode plate 11 is applied to an input terminal of the amplifier 530, the potential V12 of the electrode plate 12 is applied to another input terminal of the amplifier 530, and the amplifier 530 outputs a difference between the potential V11 of the electrode plate 11 and the potential V12 of the electrode plate 12.

Accordingly, a waveform output from the amplifier 530 is the first lead waveform I′. An output of the amplifier 530 is input to the ADC 550.

The ADC 550 converts a signal input from the amplifier 530 into a digital signal, and outputs the digital signal to the communication section 570.

The potential V12 of the electrode plate 12 is applied to an input terminal of the amplifier 540, the potential V13 of the electrode plate 13 is applied to another input terminal of the amplifier 540, and the amplifier 540 outputs a difference between the potential V12 of the electrode plate 12 and the potential V13 of the electrode plate 13.

Accordingly, a waveform output from the amplifier 530 is the second lead waveform II′. An output of the amplifier 540 is input to the ADC 560.

The ADC 560 converts a signal input from the amplifier 540 into a digital signal, and outputs the digital signal to the communication section 570.

Accordingly, a signal output from the ADC 550 is regarded as a signal converted from the first lead waveform I′ into a digital signal, and a signal output from the ADC 560 is regarded as a signal converted from the second lead waveform II′ into a digital signal.

The communication section 570 sends the digital signals output from the ADC 550 and the ADC 560 to the electrocardiograph 400.

In the following, for convenience, the digital signals converted by the ADCs 450, 460, 550, and 560 from the first lead waveform I, the second lead waveform II, the first lead waveform I′, and the second lead waveform II′ are referred to by the same designations from before conversion: the first lead waveform I, the second lead waveform II, the first lead waveform I′, and the second lead waveform II′.

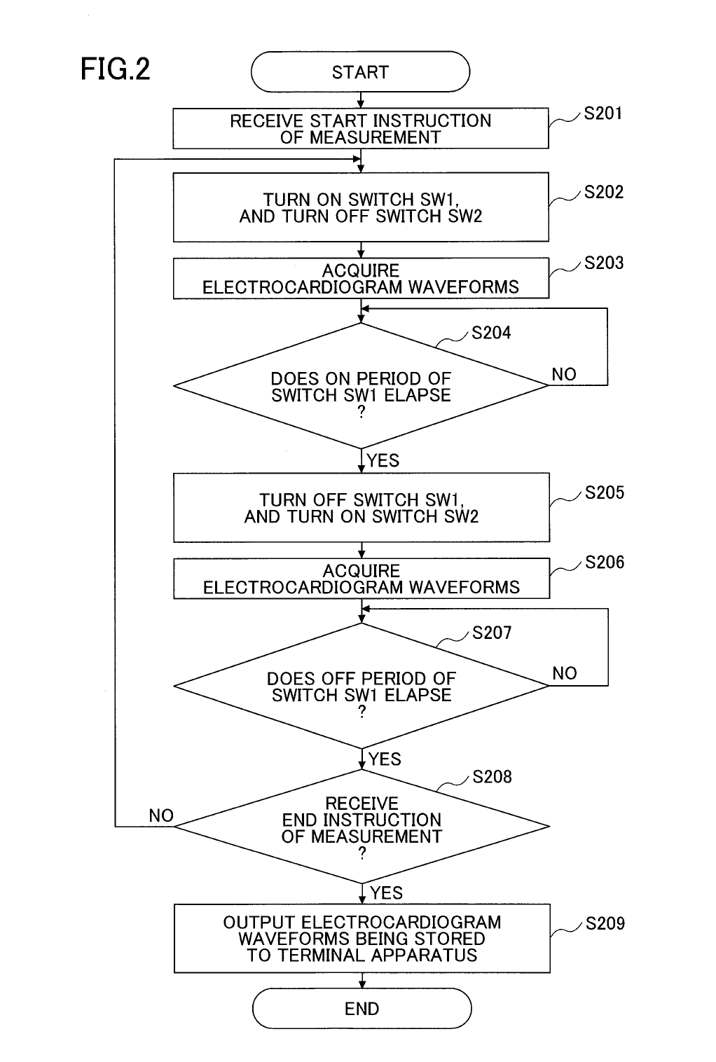

Next, an operation of the electrocardiograph system 100 of the first embodiment will be described with reference to FIG. 2. FIG. 2 is a flowchart for explaining the operation of the electrocardiograph system of the first embodiment.

In the electrocardiograph system 100 of the first embodiment, when receiving the instruction of the start for measuring the electrocardiogram waveforms (step S201), the switch SW1 is turned on by the controller 410 in the electrocardiograph 400 and the switch SW2 is turned off by the controller 510 in the electrocardiograph 500 (step S202).

Subsequently, the electrocardiograph system 100 acquires the electrocardiogram waveforms from the electrocardiograph 400 and the electrocardiograph 500, respectively (step S203).

Next, in the electrocardiograph system 100, the controller 410 of the electrocardiograph 400 determines whether the ON term-period of the switch SW1 elapses (step S204). When it is determined that the ON term-period does not elapse, the controller 410 waits for a certain interval and repeats step S204.

In the electrocardiograph system 100, when it is determined that the ON term-period elapses in step S204, the controller 410 of the electrocardiograph 400 turns off the switch SW1, and the controller 510 of the electrocardiograph 500 turns on the switch SW2 (step S205).

Subsequently, the electrocardiograph system 100 acquires the electrocardiogram waveforms from the electrocardiograph 400 and the electrocardiograph 500 (step S206).

Subsequently, in the electrocardiograph system 100, the controller 410 of the electrocardiograph 400 determines whether the OFF term-period of the switch SW1 elapses (step S207). When it is determined that the OFF term-period does not elapse, the controller 410 waits for a certain interval and repeats step S204.

Upon determining that the OFF term-period of the switch SW1 elapses in step S207, the electrocardiograph system 100 determines whether the instruction of the end of measuring the electrocardiogram waveforms is received (step S208). When it is determined that the instruction of the end is not received in step S208, the electrocardiograph system 100 goes back to step S202, turns on the switch SW1, and turns off the switch SW2.

Upon determining that the instruction of the end is received in step S208, in the electrocardiograph system 100, the output section 480 of the electrocardiograph 400 outputs the electrocardiogram waveforms stored in the memory 490, and displays the electrocardiogram waveforms at the terminal apparatus 300 (step S209). The electrocardiograph system 100 terminates this process.

The electrocardiogram waveforms to be stored in the memory 490 are the first lead waveform I, the second lead waveform II, the first lead waveform I′, and the second lead waveform II′. Moreover, the electrocardiogram waveforms to be displayed at the terminal apparatus 300 are at least two waveforms, which include one of the first lead waveform I and the first lead waveform I′, and one of the second lead waveform II and the second lead waveform II′. In the first embodiment, because priority levels of the first lead waveform I and the second lead waveform II are higher than those of the first lead waveform I′ and the second lead waveform II′, when the first lead waveform I and the second lead waveform II are measured normally, the first lead waveform I and the second lead waveform II alone may be displayed. Moreover, in the first embodiment, when the first lead waveform I and the second lead waveform II are not correctly measured, the first lead waveform I′ and the second lead waveform II′ may be displayed. That is, in the first embodiment, four waveforms including the first lead waveform I, the second lead waveform II, the first lead waveform I′, and the second lead waveform II′ may be displayed. Alternatively, a correctly measured waveform alone may be displayed. A method for determining whether the waveform is correctly measured will be described later.

In the first embodiment, for instance, a sampling period of data by the ADC 450, the ADC 460, the ADC 550, and the ADC 560 may be 1.25 KHz, such that 10 sets of data are sampled alternately by the electrocardiograph 400 and the electrocardiograph 500.

By sampling in this manner, it is possible for a roughness of the sampling to be the same as that in a case in which the sampling period is 125 Hz, for instance.

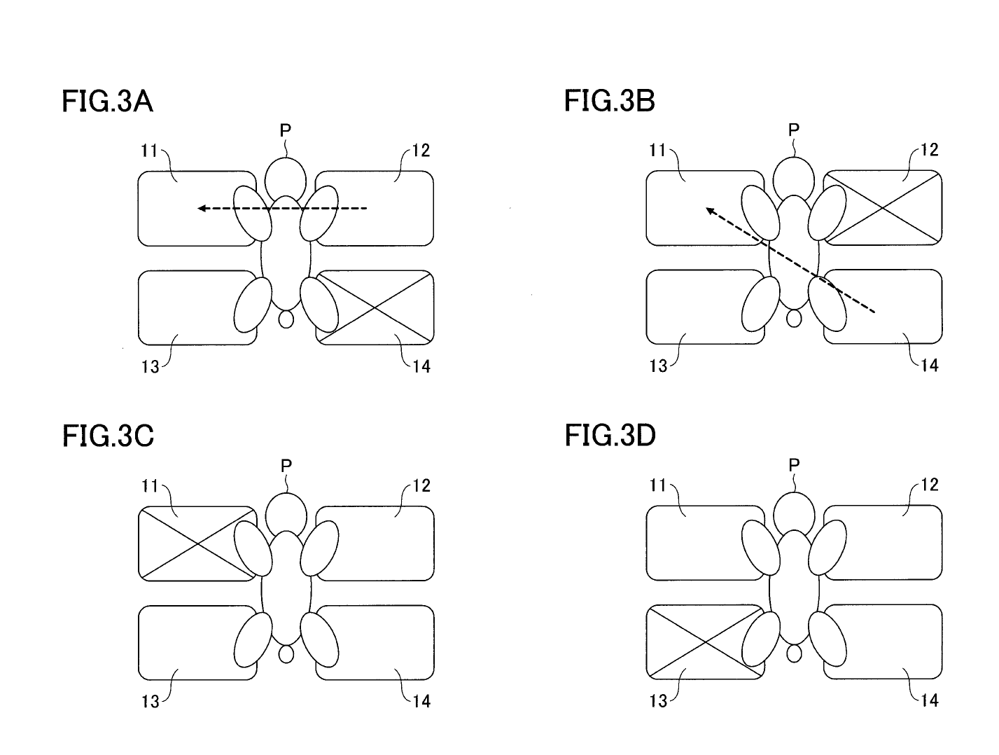

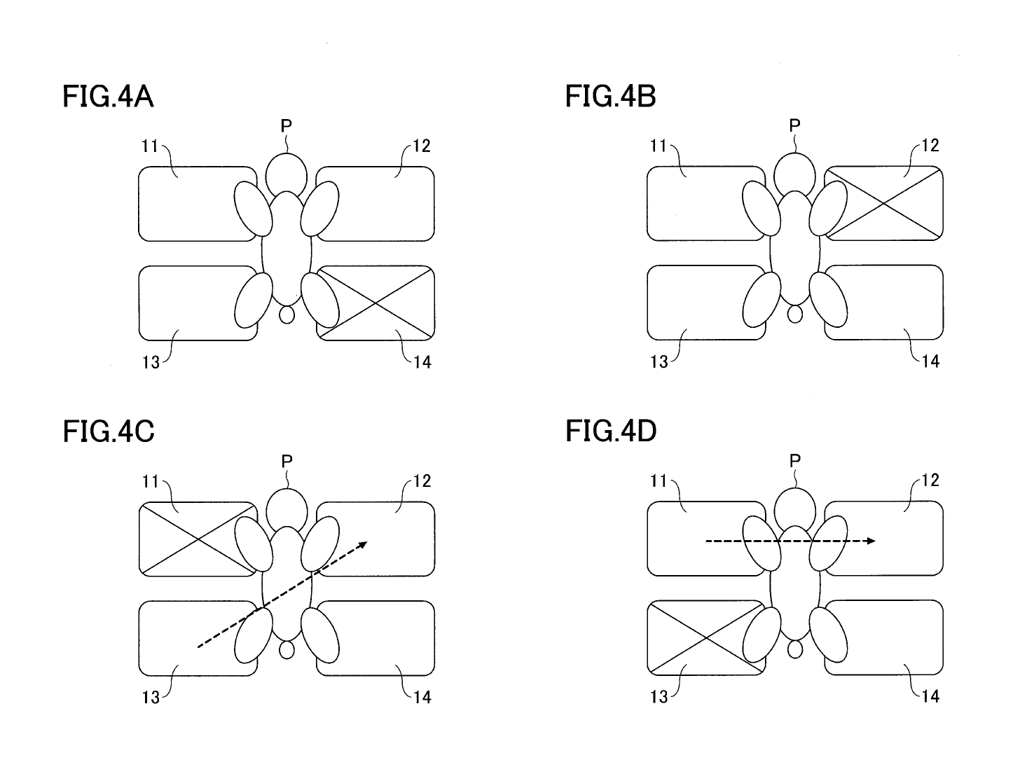

Next, a measurement of the electrocardiogram waveforms by the electrocardiograph system 100 of the first embodiment will be described with reference to FIG. 3A through FIG. 3D and FIG. 4A through FIG. 4D.

In FIG. 3A through FIG. 3D and FIG. 4A through FIG. 4D, for instance, the electrode plates 11 through 14 are deployed on an examination table, on which a left foreleg of the living body P contacts the electrode plate 11, a right foreleg contacts the electrode plate 12, a left hind leg contacts the electrode plate 13, and a right hind leg contacts the electrode plate 14.

FIG. 3A and FIG. 4A illustrate a state in which the right hind leg is away from the electrode plate 14 and the other three legs contact corresponding electrode plates 11, 12, and 13, among the four limbs of the living body P. FIG. 3B and FIG. 4B illustrate a state in which the right foreleg is away from the electrode plate 12 and the other three legs contact corresponding electrode plates 11, 13, and 14, among the four limbs of the living body P. FIG. 3C and FIG. 4C illustrate a state in which the left foreleg is away from the electrode plate 11 and the other three legs contact corresponding electrode plates 12, 13, and 14, among the four limbs of the living body P. FIG. 3D and FIG. 4D illustrate a state in which the left foreleg is away from the electrode plate 13 and the other three legs contact corresponding electrode plates 11, 12, and 14, among the four limbs of the living body P.

FIG. 3A through FIG. 3D are first diagrams for explaining the measurement of the electrocardiogram waveforms by the electrocardiograph system of the first embodiment. With reference to FIG. 3A through FIG. 3D, measurements of the first lead waveform I and the second lead waveform II by the electrocardiograph 400 will be described below. In the electrocardiograph 400, a potential is applied to the electrode plate 13 as the reference.

In a case of FIG. 3A, because the potential V14 of the electrode plate 14 is not acquired, the electrocardiograph 400 does not acquire the second lead waveform II representing the difference between the potential V11 of the electrode plate 11 and the potential V14. However, referring to FIG. 3A, because the potential V11 of the electrode plate 11 and the potential V12 of the electrode plate 12 are acquired, the first lead waveform I representing the difference between the potential V12 and the potential V11 is acquired.

In a case of FIG. 3B, because the potential V12 of the electrode plate 12 is not acquired, the electrocardiograph 400 does not acquire the first lead waveform I representing the difference between the potential V11 of the electrode plate 11 and the potential V12. However, referring to FIG. 3B, because the potential V11 of the electrode plate 11 and the potential V14 of the electrode plate 14 are acquired, the second lead waveform II representing the difference between the potential V14 and the potential V11.

In a case of FIG. 3C, because the potential V11 of the electrode plate 11 is not acquired, the electrocardiograph 400 does not acquire the first lead waveform I representing the difference between the potential V11 of the electrode plate 11 and the potential V12 nor the second lead waveform II representing the difference between the potential V11 of the electrode plate 11 and the potential V14 of the electrode plate 14 is acquired.

In a case of FIG. 3D, because the potential V13 of the electrode plate 13 that is the reference potential is not acquired, neither the first lead waveform I nor the second lead waveform II are acquired.

FIG. 4A through FIG. 4D are second diagrams for explaining the measurement of the electrocardiogram waveforms by the electrocardiograph system of the first embodiment.

In a case of FIG. 4A, because the potential V14 of the electrode plate 14 that is the reference potential in the electrocardiograph 500 is not acquired, the first lead waveform I′ and the second lead waveform II′ are not acquired.

In a case of FIG. 4B, because the potential V12 of the electrode plate 12 is not acquired, the electrocardiograph 500 does not acquire the first lead waveform I′ representing the difference between the potential V11 of the electrode plate 11 and the potential V12 nor the second lead waveform II′ representing the difference between the potential V12 of the electrode plate 12 and the potential V13 of the electrode plate 13.

In a case of FIG. 4C, because the potential V11 of the electrode plate 11 is not acquired, the electrocardiograph 500 does not acquire the first lead waveform I′ representing the difference between the potential V11 of the electrode plate 11 and the potential V12. However, referring to FIG. 4C, because the potential V13 of the electrode plate 13 and the potential V12 of the electrode plate 12 are acquired, the second lead waveform II′ representing the difference between the potential V13 and the potential V12 is acquired.

In a case of FIG. 4D, because the potential V13 of the electrode plate 13 is not acquired, the electrocardiograph 500 does not acquire the second lead waveform II′ representing the difference between the potential V13 of the electrode plate 13 and the potential V12. However, referring to FIG. 4D, because the potential V11 of the electrode plate 11 and the potential V12 of the electrode plate 12 are acquired, the first lead waveform I′ representing the difference between the potential V11 and the potential V12 is acquired.

According to the first embodiment, if three out of four legs of the living body P contact corresponding electrode plates, it is possible to acquire at least one electrocardiogram waveform by one lead. In general, it is considered that a quadruped animal will occasionally stand on three legs; however, it is considered rare for such an animal to stand on two legs, even if the animal stands up, it is only for a short time.

As described above, according to the first embodiment, even in a case in which not all four limbs do contact corresponding electrode plates, it is possible to measure the electrocardiogram waveforms of the living body P. Also, according to the first embodiment, because the electrocardiogram waveforms of the living body P are measured in the time sharing manner by a plurality of electrocardiographs in which the reference potential is applied to different electrode plates, it is possible to measure the electrocardiogram waveforms even if a leg of the living body P is away from one of the electrode plates set as the reference in the plurality of electrocardiographs.

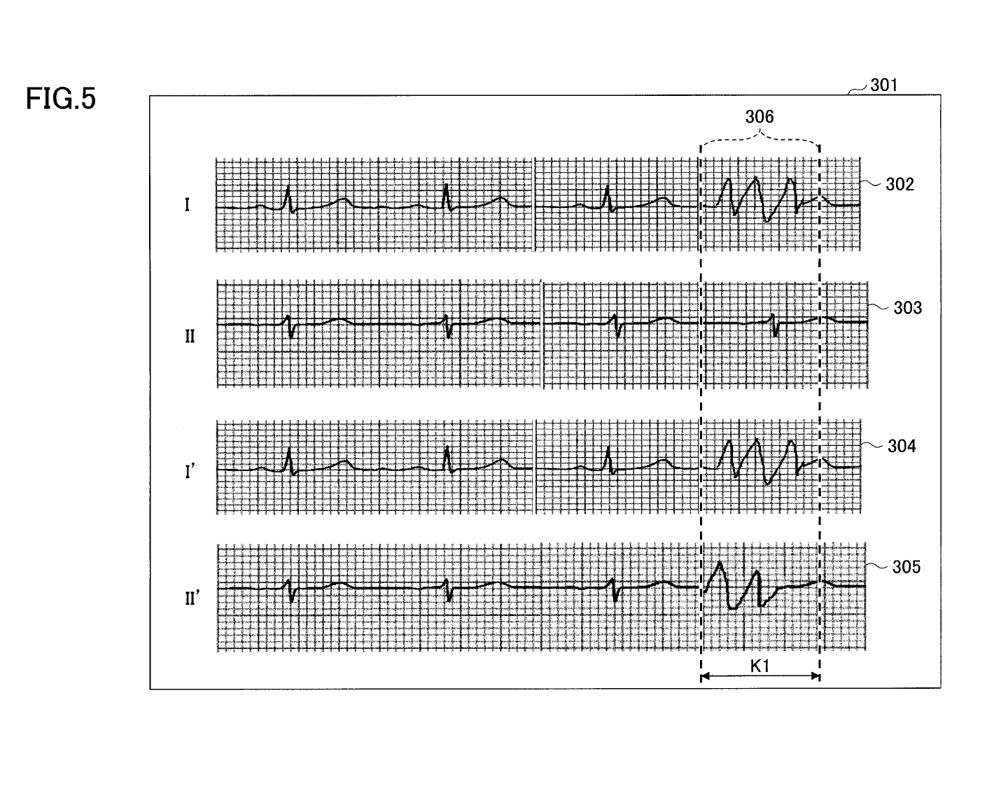

FIG. 5 is a diagram illustrating a display example of the electrocardiograph system of the first embodiment. For instance, a screen 301 in FIG. 5 illustrates an example of the electrocardiogram waveforms of the living body P displayed at the terminal apparatus 300.

On the screen 301, a waveform 302, a waveform 303, a waveform 304, and a waveform 305 are displayed. The waveform 302 represents the first lead waveform I, the waveform 303 represents the second lead waveform II, the waveform 304 represents the first lead waveform I′, and the waveform 305 represents the second lead waveform II′.

In this example of the screen 301, the waveforms 302, 304, and 305 are disturbed in a term K1. However, in the waveform 303, no disturbance of the waveform occurs during the term K1.

Accordingly, in this case, it is observed that the second lead waveform II of the living body P alone is measured normally. That is, in the first embodiment, even in a case in which one of the legs (for example, the left foreleg) of the living body P does not contact with the electrode plate 11, it is possible to measure the electrocardiogram waveforms of the living body P.

As described above, according to the first embodiment, it is possible to reduce effects on the electrocardiogram waveforms depending on a contact state between the electrode plates and the living body.

In the first embodiment, as an example, the living body P is a quadruped animal; however, the living body P is not limited to this example.

For instance, the living body P may be an infant of a few months old who is not able to stand up and walk. As another example, the living body P may be an adult or the like who may be in a state in which one or more limbs are not contacted to one or more corresponding electrodes among the four limbs.

Moreover, in the first embodiment, the electrocardiograph 200 includes the electrocardiograph 400 in which the potential V13 of the electrode plate 13 is set as the reference potential, and the electrocardiograph 500 in which the potential V14 of the electrode plate 14 is set as the reference potential; however, a configuration of the electrocardiograph 200 is not limited to this configuration.

For instance, the electrocardiograph 200 may further include an electrocardiograph in which the potential V11 of the electrode plate 11 is set as the reference potential, and an electrocardiograph in which the potential V12 of the electrode plate 12 is set as the reference potential, in addition to the electrocardiograph 400 and the electrocardiograph 500.

As described above, in a case of including four electrocardiographs in the electrocardiograph 200, if three electrode plates contact limbs of the living body P among four electrode plates, it is possible to acquire the electrocardiogram waveforms by at least two leads. Hence, it is possible to reduce effects on the electrocardiogram waveforms due to the contact state between the electrode plates and the living body P.

### Second Embodiment

In the following, a second embodiment will be described with reference to drawings. Different from the first embodiment, in the second embodiment, there is notification of electrocardiogram waveforms being acquired in a state in which a leg of the living body P does not contact an electrode plate. Accordingly, in the following description of the second embodiment, differences from the first embodiment are described, and components that are functionally equivalent to that in the first embodiment are designated by the same reference numerals, and the description thereof is omitted.

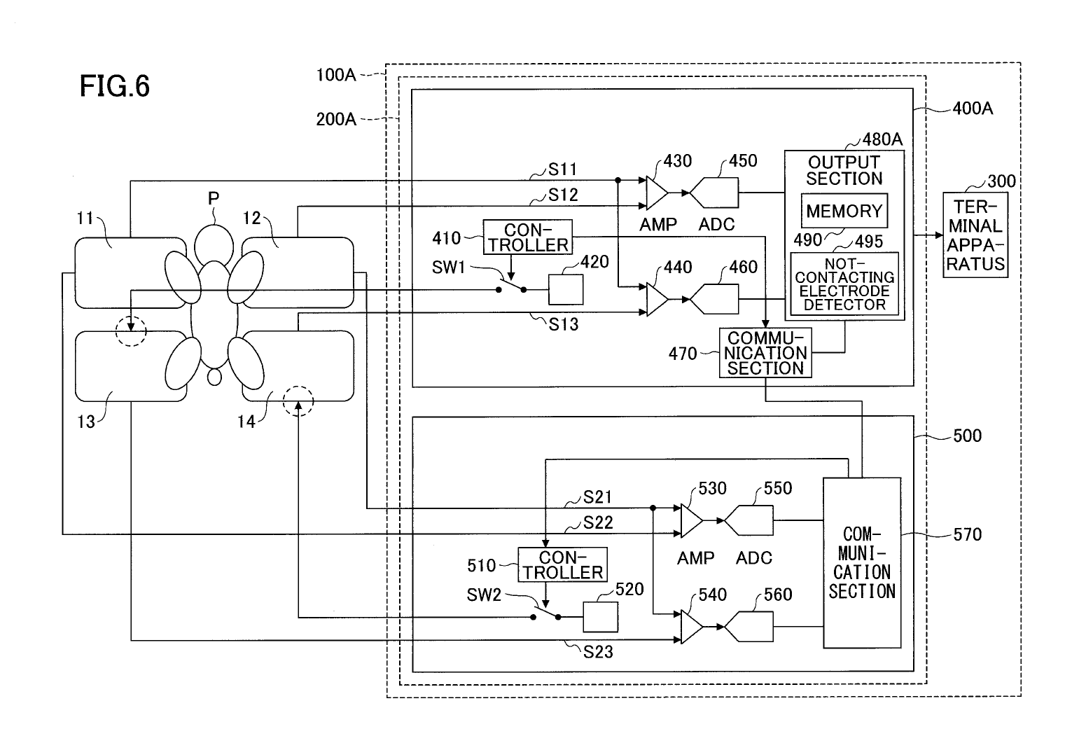

FIG. 6 is a diagram for explaining an electrocardiograph system of the second embodiment. An electrocardiograph system 100A of the second embodiment includes an electrocardiograph 200A and the terminal apparatus 300.

The electrocardiograph 200A of the second embodiment includes an electrocardiograph 400A and the electrocardiograph 500. The electrocardiograph 400A of the second embodiment includes the controller 410, the driving section 420, the amplifiers 430 and 440, the ADC 450 and 460, the communication section 470, an output section 480A, and the switch SW1.

An output section 480A of the second embodiment includes the memory 490, and a not-contacting electrode detector 495. The not-contacting electrode detector 495 of the second embodiment detects, from the electrocardiogram waveforms stored in the memory 490, an electrocardiogram waveform measured in a state in which a limb of the living body P does not contact a corresponding electrode plate among the electrode plates 11 through 14.

More specifically, the not-contacting electrode detector 495 specifies an electrocardiogram waveform measured in any one of states depicted in FIG. 3A through FIG. 3D and FIG. 4A through FIG. 4D, from among the first lead waveform I, the second lead waveform II, the first lead waveform I′, and the second lead waveform II′ stored in the memory 490. In the following description, the electrocardiogram waveform measured in any one of states depicted in FIG. 3A through FIG. 3D and FIG. 4A through FIG. 4D is called a “not-contacting electrode waveform”.

Moreover, the not-contacting electrode detector 495 specifies a term when the living body P does not contact the electrode plate, in the not-contacting electrode waveform. In the following description, the term when the living body P does not contact the electrode plate is called a “not-contacting electrode term”. A method for specifying the not-contacting electrode term by the not-contacting electrode detector 495 will be described later.

Moreover, when outputting the electrocardiogram waveforms to the terminal apparatus 300, the output section 480A of the second embodiment outputs a detection result of the not-contacting electrode detector 495 with the electrocardiogram waveforms, to be displayed by the terminal apparatus 300.

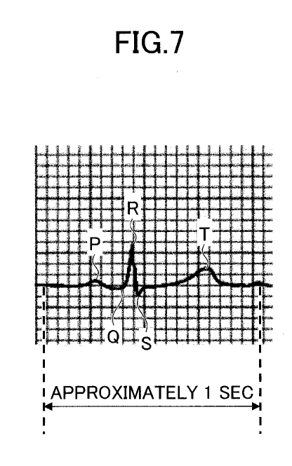

In the following, the method for specifying the not-contacting electrode term by the not-contacting electrode detector 495 will be described with reference to FIG. 7. FIG. 7 is a diagram illustrating an example of a normal electrocardiogram waveform.

The normal electrocardiogram waveform is mainly formed by a P wave, a Q wave, an R wave, an S wave, and a T wave for each heartbeat.

The not-contacting electrode detector 495 of the second embodiment retains a threshold for an amplitude of the R wave, and specifies an electrocardiogram waveform in which the amplitude of the R wave exceeds the threshold, as the not-contacting electrode waveform, from among the electrocardiogram waveforms stored in the memory 490.

The not-contacting electrode detector 495 of the second embodiment further specifies a term where the amplitude of the R wave is greater than the threshold in the not-contacting electrode waveform, as the not-contacting electrode term.

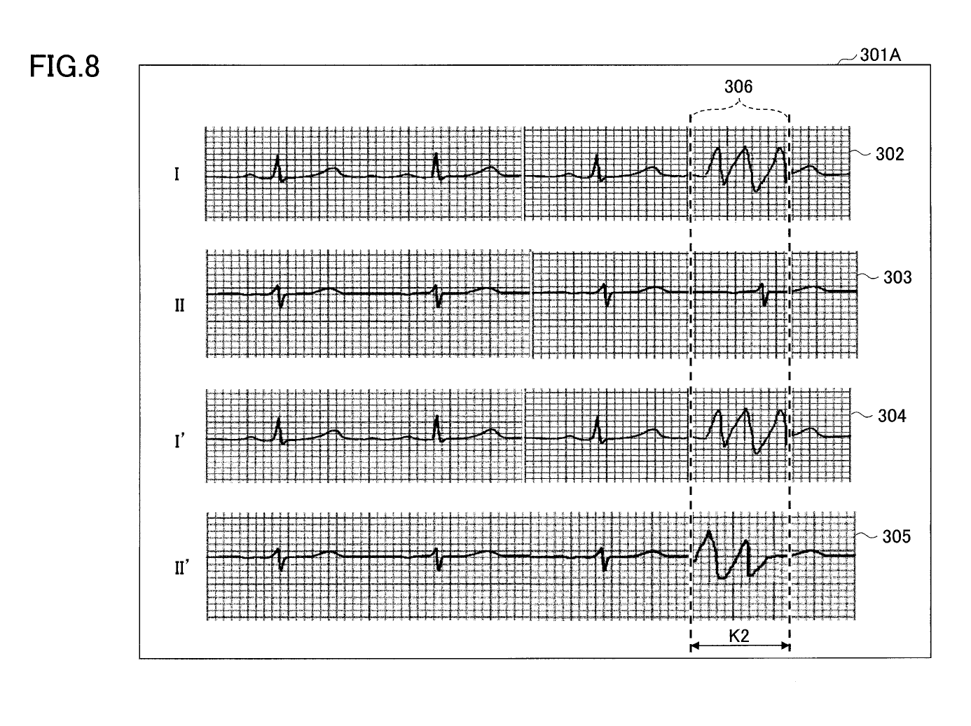

FIG. 8 is a diagram illustrating a display example of the electrocardiograph system of the second embodiment. In a screen 301A depicted in FIG. 8, a term K2 is specified as the not-contacting electrode term, and a marker 306 is displayed to indicate that the term K2 is the not-contacting electrode term.

In the second embodiment, as described above, the not-contacting electrode term may be specified in the electrocardiograph 200A, and the specified term may be displayed at the terminal apparatus 300.

In the second embodiment, the not-contacting electrode term is specified, and the specified term is displayed with the electrocardiogram waveforms at the terminal apparatus 300; however, the second embodiment is not limited to this manner.

In the second embodiment, for instance, an electrocardiogram waveform specified as the not-contacting electrode waveform is not displayed at the terminal apparatus 300. For instance, referring to the example depicted in FIG. 8 with respect to the term K2, the waveforms 302, 304, and 305 are regarded as the not-contacting electrode waveform having the R wave of which the amplitude becomes greater than the threshold. Accordingly, in this case, the electrocardiograph system 100A does not display the waveforms 302, 304, and 305 at the terminal apparatus 300, and the waveform 303 (the second lead waveform II) alone may be displayed at the terminal apparatus 300.

Moreover, in the second embodiment, for instance, the electrocardiogram waveforms other than the not-contacting electrode waveform may be displayed in a different display method from that for displaying the not-contacting electrode waveform. Specifically, for instance, the electrocardiogram waveforms other than the not-contacting electrode waveform may be displayed with more emphasis than the not-contacting electrode waveform. Alternatively, a message or the like may be displayed to inform that a not-contacting electrode waveform is included.

Moreover, in the electrocardiograph system 100A of the second embodiment, the electrocardiograph 400A of the electrocardiograph 200A includes the not-contacting electrode detector 495; however, the configuration is not limited to this example. For instance, the not-contacting electrode detector 495 may be provided in the terminal apparatus 300.

As described above, according to the second embodiment, an electrocardiogram waveform measured in a state in which the electrode plate does not contact the living body P, and the term K2 are specified and reported. Hence, according to the second embodiment, it is possible for a person observing electrocardiogram waveforms displayed at the terminal apparatus 300 to provide a normal electrocardiogram waveform.

### Third Embodiment

In the following, a third embodiment will be described with reference to drawings. In the third embodiment, the method for specifying the not-contacting electrode waveform is different from that in the second embodiment. Hence, in the following description of the third embodiment, differences from the second embodiment will be described, and components that are functionally equivalent to that in the second embodiment are designated by the same reference numerals, and the description thereof is omitted.

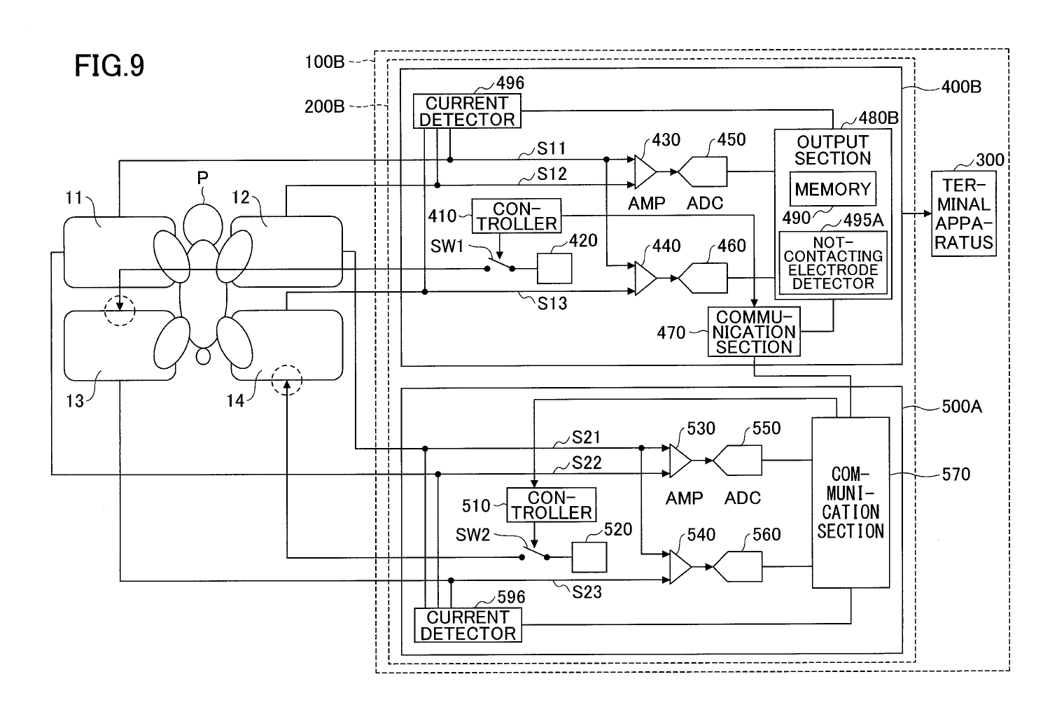

FIG. 9 is a diagram for explaining the electrocardiograph system of the third embodiment. An electrocardiograph system 100B includes an electrocardiograph 200B and the terminal apparatus 300.

The electrocardiograph 200B includes an electrocardiograph 400B and an electrocardiograph 500A. The electrocardiograph 400B of the third embodiment includes the controller 410, the driving section 420, the amplifiers 430 and 440, the ADC 450 and 460, the communication section 470, an output section 480B, a current detector 496, and the switch

SW1.

The output section 480B of the third embodiment includes the memory 490, and a not-contacting electrode detector 495A. In the electrocardiograph 400B, the not-contacting electrode detector 495A specifies the not-contacting electrode waveform depending on whether currents from the electrode plates 11, 12, and 14 other than the electrode plate 13 being the reference are detected.

The current detector 496 is connected to each of the signal lines S11, S12, and S13. The signal line S11 connects between the electrode 11 and the amplifier 430, the signal line S12 connects between the electrode 11 and the amplifier 430, and the signal line S13 connects between the electrode 14 and the amplifier 440.

The current detector 496 applies weak current to each of the electrode plates 11, 12, and 14, and detects currents flowing through the signal lines S11, S12, and S13. In this case, the current detector 496 may include a current source that applies the weak current. Moreover, the current detector 496 sends a notification indicating a signal line on which the current is not detected to the not-contacting electrode detector 495A of the output section 480B.

The not-contacting electrode detector 495A specifies an electrode plate corresponding to a signal line at which current is not detected by the current detector 496, as the electrode plate is not contacting the living body P. Then, the not-contacting electrode detector 495A specifies the electrocardiogram waveform, which is output from an amplifier connected to a signal line on which the current is not detected, as the not-contacting electrode waveform.

The electrocardiograph 500A of the third embodiment includes the controller 510, the driving section 520, the amplifiers 530 and 540, the ADC 550, the ADC 560, the communication section 570, a current detector 596, and the switch SW2.

The current detector 596 is connected to each of the signal lines S21, S22, and S23. The signal line S21 connects between the electrode plate 12 and the amplifier 530, the signal line S22 connects between the electrode plate 11 and the amplifier 530, and the signal line S23 connects between the electrode plate 13 and the amplifier 540.

The current detector 596 applies the current to each of the electrode plates 11, 12, and 13, and detects the current flowing through each of the signal lines S21, S22, and S23. Moreover, the current detector 596 sends a notification indicating a signal line on which the current is not detected, to the not-contacting electrode detector 495A of the output section 480B through the communication section 570.

For instance, it is assumed that the right foreleg of the living body P does not contact the electrode plate 12. In this case, the current is not detected from the signal line S12 connecting between the electrode plate 12 and the amplifier 430, and the signal line S21 connecting between the electrode plate 12 and the amplifier 530.

Accordingly, in the electrocardiograph 400B, the current detector 496 sends a notification indicating the signal line S12 to the not-contacting electrode detector 495A. Moreover, in the electrocardiograph 500A, the current detector 596 sends a notification indicating the signal line S21 to the not-contacting electrode detector 495A.

The not-contacting electrode detector 495A specifies, from these notifications, the first lead waveform I, the first lead waveform I′, and the second lead waveform II′, which are acquired by using a potential of the electrode plate 12 to which the signal line S12 and the signal line S21 are connected, as the not-contacting electrode waveform.

As described above, according to the third embodiment, it is possible for a person observing the electrocardiogram waveforms displayed at the terminal apparatus 300 to provide the normal electrocardiogram waveforms.

Also, it is possible to reduce effects on the electrocardiogram waveforms due to the contact state between the electrode plates and the living body P.

Although the present invention has been described based on the respective embodiments, the present invention is not limited to requirements described in the above embodiments. Regarding these points, it is possible to change the scope of the present invention within the scope not to obscure it, and requirements can be appropriately determined according to an application form.

What is claimed is

1. An electrocardiograph, comprising:
a first electrocardiograph configured to measure a first electrocardiogram waveform with a potential of a first electrode being set as a reference potential among a plurality of electrodes; and
a second electrocardiograph configured to measure a second electrocardiogram waveform with a potential of a second electrode being set as the reference potential among the plurality of electrodes,
wherein the first electrocardiograph and the second electrocardiograph are switched in a time sharing manner, and the first electrocardiogram waveform and the second electrocardiogram waveform are measured.

2. The electrocardiograph as claimed in claim 1,
wherein the first electrocardiograph includes
a first driving section configured to apply the reference potential to the first electrode; and
a first controller configured to control a connection and a disconnection between the first electrode and the first driving section,
wherein the second electrocardiograph includes
a second driving section configured to apply the reference potential to the second electrode; and
a second controller configured to control a connection and a disconnection between the second electrode and the second driving section, and
wherein the first controller is configured to
control the second controller to disconnect between the second electrode and the second driving section upon connecting between the first electrode and the first driving section; and
control the second controller to connect between the second electrode and the second driving section upon disconnecting between the first electrode and the first driving section.

3. The electrocardiograph as claimed in claim 1,
wherein the plurality of electrodes are four electrodes,
wherein the first electrocardiogram waveform includes one of a first lead waveform and a second lead waveform, among limb 6-lead waveforms induced by the four electrodes with the first electrode being the reference, and
wherein the second electrocardiogram waveform includes one of a first lead waveform and a second lead waveform, among limb 6-lead waveforms induced by the four electrodes with the second electrode being the reference.

4. The electrocardiograph as claimed in claim 1,
wherein the first electrocardiograph and the second electrocardiograph are able to communicate with each other,
wherein the second electrocardiograph sends the second electrocardiogram waveform to the first electrocardiograph, and
wherein the first electrocardiograph includes
a storage section configured to store the first electrocardiogram waveform and the second electrocardiogram waveform, and
an output section configured to output the first electrocardiogram waveform and the second electrocardiogram waveform to a terminal apparatus.

5. The electrocardiograph as claimed in claim 4, wherein the output section is configured to
specify an electrocardiogram waveform in which an amplitude of a certain waveform included in the first electrocardiogram waveform and the second electrocardiogram waveform exceeds a threshold, and
output a notification indicating the specified electrocardiogram waveform.

6. The electrocardiograph as claimed in claim 4,
wherein the first electrocardiograph includes
a first current detector configured to supply a current with respect to electrodes other than the first electrode among the plurality of electrodes, and to detect an electrode in which the current does not flow,
wherein the second electrocardiograph includes
a second current detector configured to supply a current with respect to electrodes other than the second electrode among the plurality of electrodes, and to detect an electrode in which the current does not flow, and
wherein the output section specifies the first electrocardiogram waveform and the second electrocardiogram waveform where are measured by using one or more of the electrodes detected by the first current detector and the second current detector, and outputs a notification indicating a specified electrocardiogram waveform.

7. An electrocardiograph system, comprising:
an electrocardiograph; and
a terminal apparatus,
wherein the electrocardiograph includes
a first electrocardiograph configured to measure a first electrocardiogram waveform with a potential of a first electrode being as a reference potential among a plurality of electrodes, and
a second electrocardiograph configured to measure a second electrocardiogram waveform with a potential of a second electrode being as the reference potential among the plurality of electrodes, and
wherein the terminal apparatus is configured to
display the first electrocardiogram waveform and the second electrocardiogram waveform, which are output from the electrocardiograph.
